(19) United States
(12) Patent Application Publication
(10) Pub. No.: US 2004/0006434 A1
(43) Pub. Date: Jan. 8, 2004

(54) Method for the identification of essential and conditional essential genes

(75) Inventors: Ian G. Charles, London (GB); Andrew Allen, London (GB); Duncan J. Maskell, Cambridge (GB)

Correspondence Address: NIXON & VANDERHYE, PC, 1100 N GLEBE ROAD, 8TH FLOOR, ARLINGTON, VA, 22201-4714, US

(73) Assignee: ARROW THERAPEUTICS LIMITED

(21) Appl. No.: 10/356,733

(22) Filed: Feb. 3, 2003

Related U.S. Application Data
(63) Continuation-in-part of application No. 10/031,786, filed on Mar. 21, 2002.
Application No. 10/031,786 is a 371 of international application No. PCT/GB00/02879, filed on Jul. 26, 2000.
(60) Provisional application No. 60/352,858, filed on Feb. 1, 2002.

(30) Foreign Application Priority Data
Jul. 26, 1999 (GB) 9917531.7

(51) Int. Cl. C12Q 1/68; G06F 19/00; G01N 33/48; G01N 33/50; C12P 19/34

(52) U.S. Cl. 702/20; 435/6; 435/91.2

(57) ABSTRACT

A method for identifying an essential gene of an organism, which method comprises: (i) providing a library of transposon insertion mutants of the said organism, wherein the transposon comprises an RNA polymerase recognition sequence; (ii) isolating chromosomal DNA from the library of (i), (iii) digesting the chromosomal DNA with a restriction endonuclease that is capable of cutting 5′ of the RNA polymerase recognition site in the transposon and 3′ of the RNA polymerase recognition site in the chromosomal DNA flanking the transposon; (iv) self-ligating the digested DNA, (v) amplifying the self-ligated DNA by inverse PCR (iPCR), (vi) transcribing RNA from the amplified DNA, (vii) hybridising the transcribed RNA with an oligonucleotide array; and (viii) identifying a probe on the oligonucleotide array which corresponds to an essential gene of the organism. Genes identified in such a method are useful as substrates for use in screens for antibacterials, antiparasitics, fungicides, pesticides and herbicides.

### FIELD OF THE INVENTION

This invention relates to a method for the identification of essential and conditional essential genes, in particular in bacteria. The invention also relates to antibacterials, fungicides, antiparasitics, pesticides and herbicides.

### BACKGROUND OF THE INVENTION

The increase in prevalence of antibiotic-resistant bacteria, for example, has renewed interest in the search for new targets for antibacterial agents. Essential genes and in particular the proteins which they encode may be good substrates for use in screens for antibacterials, antiparasitics, fungicides, pesticides and herbicides. Essential genes and their protein products potentially represent such targets.

Additionally, there is an interest in the identification of conditional essential genes, that is genes which are essential for the survival of an organism in a particular environment. In the case of pathogenic bacteria, for example, conditional essential genes include those which are required for survival in a host. Such genes and the proteins which they encode may also be good targets for use in screens for antibacterials. In particular, bacteria which carry mutations in such genes may be useful in attenuated live vaccines.

### SUMMARY OF THE INVENTION

Essential genes are those genes which, when missing (eg. because of a chromosomal deletion) or mutated to render them non-functional, result in a lethal phenotype That is, genes without which an organism cannot survive. Conditional essential genes are those genes which are not although not absolutely essential for the survival of an organism under all conditions, are nevertheless essential for survival under various conditional restraints. Examples of particular conditional restraints include survival at elevated temperatures and survival of a pathogen within its host.

We have devised a general method for the identification of essential and conditional essential genes in the genome of an organism using a transposon-mediated technique. We have called the technique Transposon Mediated Differential Hybridisation (TMDH). The technique may also be used for the identification of the conditional essential genes in the genome of an organism subject to a particular conditional restraint.

According to the present invention there is thus provided a method for the identification of an essential gene of an organism, which method comprises:

(i) providing a library of transposon insertion mutants of the said organism, wherein the transposon comprises an RNA polymerase recognition sequence;

(ii) isolating chromosomal DNA from the library of (i);

(iii) digesting the chromosomal DNA with a restriction endonuclease that is capable of cutting 5′ of the RNA polymerase recognition site in the transposon and 3′ of the RNA polymerase recognition site in the chromosomal DNA flanking the transposon;

(iv) self-ligating the digested DNA;

(v) amplifying the self-ligated DNA by inverse PCR (iPCR);

(vi) transcribing RNA from the amplified DNA;

(vii) hybridising the transcribed RNA with an oligonucleotide array; and

(viii) identifying a probe on the oligonucleotide array which corresponds to an essential gene of the organism.

The invention also provides:

a method for identifying a conditional essential gene of an organism, which method comprises:

(a) providing a first sample of a library of transposon insertion mutants of the said organism (input library);

(b) providing a second sample of the library and subjecting that sample to a conditional restraint;

(c) collecting the mutants that survive the conditional restraint in step (ii) to give a second library (output library);

(d) carrying out a method according to steps (ii) to (vi) set out in the method above on the input library from step (a) and on the output library from step (c);

(e) hybridising the transcribed RNA derived from the input library and from the output library to the same or different oligonucleotide arrays; and

(f) identifying a probe on the oligonucleotide array(s) which corresponds to a conditional essential gene of the organism;

a method for identifying:

(i) an inhibitor of transcription and/or translation of an essential gene or a conditional essential gene identified by a method of the invention; and/or

(ii) an inhibitor of activity of a polypeptide encoded by a said gene, which method comprises determining whether a test substance can inhibit transcription and/or translation of a said gene and/or activity of a polypeptide encoded by a said gene;

an inhibitor identified by such a method;

an inhibitor of the invention, wherein the essential or conditional essential gene is from a bacterium, fungus or eukaryotic parasite;

a pharmaceutical composition comprising such an inhibitor and a pharmaceutically acceptable carrier or diluent;

a method of treating a host suffering from a bacterial, fungal or eukaryotic parasite infection, which method comprises the step of administering to the host a therapeutically effective amount of such an inhibitor;

an inhibitor of the invention, wherein the essential or conditional essential gene is from a bacterium, fungus or pest;

a method of treating a bacterial, fungal or plant pest infection of a plant, which method comprises the step of administering to the plant an effective amount of such an inhibitor;

an inhibitor of the invention, wherein the essential or conditional essential gene is a plant conditional or essential gene;

a method of inhibiting the growth of a plant, which method comprises the step of administering to the plant an effective amount of such an inhibitor;

a method for identifying a conditional essential gene of an organism, wherein the organism is a bacterium and the conditional restraint is growth of that bacterium in its host;

a bacterium attenuated by a non-reverting mutation in one or more genes identified by such a method;

a vaccine comprising a bacterium of the invention and a pharmaceutically acceptable carrier or diluent;

a method of raising an immune response in a mammalian host, which method comprises the step of administering to the host a bacterium of the invention;

a method for the preparation of a pharmaceutical composition, which method comprises:

(a) identifying: (i) an inhibitor of transcription and/or translation of an essential gene or a conditional essential gene, and/or (ii) an inhibitor of activity of a polypeptide encoded by a said gene, by the method set out above;

(b) synthesizing an inhibitor identified in step (a); and

(c) formulating the synthesized inhibitor with a pharmaceutically acceptable carrier or diluent;

a method of treating a host suffering from a bacterial, fungal or eukaryotic parasite infection, which method comprises:

(a) identifying: (i) an inhibitor of transcription and/or translation of an essential gene or a conditional essential gene; and/or (ii) an inhibitor of activity of a polypeptide encoded by a said gene, by the method set out above;

(b) synthesizing an inhibitor identified in step (a);

(c) formulating the synthesized inhibitor with a pharmaceutically acceptable carrier or diluent; and

(d) administering to the host a therapeutically effective amount of the inhibitor formulated in (c);

a method for the preparation of a vaccine, which method comprises:

(a) identifying a conditional essential gene by the method set out above;

(b) preparing a bacterium which comprises a non-reverting mutation in a conditional essential gene identified in step (a); and

(c) formulating the bacterium prepared in step (b) with a pharmaceutically acceptable carrier or diluent; and

a method of raising an immune response in a mammalian host, which method comprises:

(a) identifying a conditional essential gene by the method set out above;

(b) preparing a bacterium which comprises a non-reverting mutation in a conditional essential gene identified in step (a);

(c) formulating the bacterium prepared in step (b) with a pharmaceutically acceptable carrier or diluent; and

(d) administering to the host a bacterium formulated in step (c).

### BRIEF DESCRIPTION OF THE FIGURES

FIG. 1 sets out the nucleotide sequence of the Tn5kan-T7 transposon (SEQ ID NO: 1). The underlined sequences correspond to the Mosaic Ends (ME) of the transposon, also referred to as insertion sequences (IS). The sequence in bold represents a T7 RNA polymerase binding site. The kanamycin resistance gene marker is highlighted in italics. The binding sites for the two iPCR oligonucleotides are outlined.

FIG. 2 shows a diagrammatic representation of target generation from Tn5kan-T7 mutant DNA. Box 1 depicts a Tn5kan-T7 transposon integrated into E. coli MG1655 chromosomal DNA, with the T7 promoter located adjacent to the ME element at the insertion site. The small arrows indicate the positions of the iPCR oligonucleotide binding sites. Box 2 depicts the chromosomal DNA following restriction digestion. Box 3 depicts the result of self-ligation of this DNA. Box 4 depicts the result of iPCR amplification of the DNA encoding the T7 promoter and DNA flanking the site of transposition. Box 5 represents the iPCR product following restriction digestion with the same enzyme used in box 2. Box 6 depicts the generation of labelled RENA from an IVT reaction from this digested iPCR product.

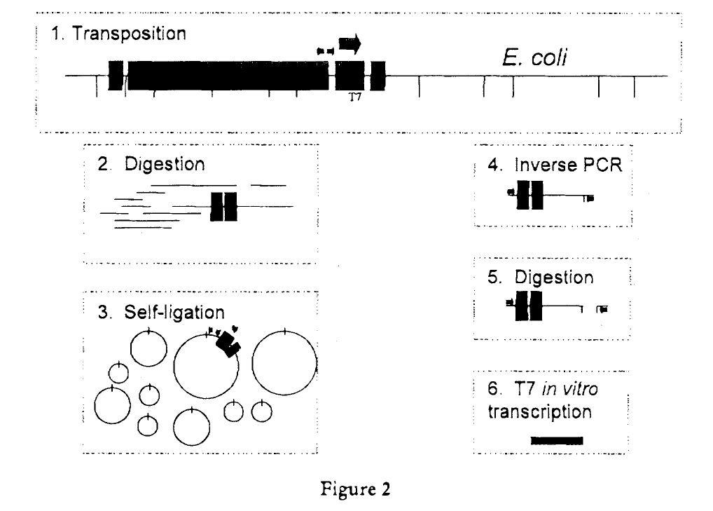

FIG. 3 shows the graphical output file generated following TMDH using DNA from the transposon mutant library digested with HaeIII, with subsequent RNA generation and labelling (as described in FIG. 2) following hybridisation onto a high-density oligonucleotide micro-array. This sub-set array design incorporates probes spanning the full-length of genes known to be essential or non-essential to E. coli viability. This figure unambiguously identifies those probes corresponding to kdtA, kdtB, and dfp as failing to hybridise to target generated from a saturating Tn5kan-T7 mutant library of E. coli MG1655. These data support the hypothesis that these genes cannot be disrupted by transposon insertion, and are likely to be essential for viability.

FIG. 4 shows the graphical output following bioinformatic feature extraction of TMDH data. Data from the locus incorporating kdtA and kdtB, (known essential genes), following the TMDH protocol using (a) HaeIII, (b) HhaI and (c) Sau3Al are displayed. Both the genes kdtA and kdtB are transcribed from the positive strand of the MG1655 chromosome while the other genes are transcribed in the reverse direction. Genes are indicated as horizontal bars. Horizontal lines above the genes indicate the position of sense probes while those below genes indicate the position of antisense probes. Above or below their respective probes are the relative fluorescence signal intensities, as a result of Cy5-labelled target RNA hybridisation. Horizontal dotted lines represent the location of Sau3AI, HaeIII, and HhaI restriction sites within the genes, respectively. Lack of hybridisation signals observed for probes designed to kdtA and kdtB from TMDH resulting from Sau3AI and HhaI digestions are consistent with these genes being classed as essential. HaeIII generated target also failed to provide any strong internal signals to the kdtA-derived probes. The two probes to kdtB that do generate a signal with HaeIII generated target (at the mutM junction) can be explained as the generation of overlapping target generated from a transposition event in the non-essential mutM gene.

### DESCRIPTION OF THE SEQUENCE LISTING

SEQ ID NO: 1 sets out the sequence of the Tn5kan-T7 transposon.

SEQ ID NO: 2 sets out the sequence of primer 67, which was used in the construction of the Tn5kan-T7 transposon.

SEQ ID NO: 3 sets out the sequence of primer 68, which was used in the construction of the Tn5kan-T7 transposon.

SEQ ID NO: 4 sets out the sequence of primer 47, which can be used to carry out iPCR in the TMDH protocol.

SEQ ID NO: 5 sets out the sequence of primer 48, which can be used to carry out iPCR in the TMDH protocol.

### DETAILED DESCRIPTION OF THE INVENTION

The invention provides a method for identifying essential and conditional essential genes of an organism. The method is a transposon insertion based method and is suitable for genome-wide analysis. If a transposon inserts into a gene which is essential and renders that gene inactive, it will not be possible to recover mutants in that gene. On the other hand, if a transposon inserts into a gene which is non-essential and renders that gene inactive, it will be possible to generate mutants carrying a transposon in that gene. In the method of the invention, an analysis of the mutants which carry transposons allows genes which are not susceptible to insertion to be identified. The method of the invention is termed Transposon Mediated Differential Hybridisation (TMDH).

In a method for the identification of an essential gene of an organism, a library of transposon insertion mutants of a particular organism is constructed. The library of transposon mutants may be used to generate RNA target sequences which comprise a complex pool of RNAs derived from polynucleotide sequences from the mutants in the library. The RNA target sequences are generated by transcribing polynucleotide sequences which flank the transposon insertion sites in the library and therefore which comprise sequence from genes that are non-essential.

The RNA target sequences are generated by: isolating fragments of DNA sequences flanking the transposons in the library; self-ligating those DNA sequences; amplifying the self-ligated DNA sequences, and transcribing the flanking sequences.

The RNA target sequences are hybridized with an oligonucleotide array derived from all or part of the genome of the organism used to generate the transposon insertion mutant library. Probes in the oligonucleotide array that do not hybridise with the RNA target sequences represent sequences in the organism which are not susceptible to transposon insertion, i.e. may correspond to genes that are essential for the survival of the organism in question. Also, probes in the oligonucleotide array which hybridise only weakly with the RNA target sequences may also represent sequences in the organism which are not susceptible to transposon insertion and which may therefore correspond to essential gene sequences.

Construction of a Library of Transposon Mutants

In the method of the invention, a library of transposon insertion mutants is generated. Transposons, sometimes called transposable elements, are mobile polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ ends of RNA transcripts with 5′ ends often truncated. Some types of virus also integrate into the host genome, for example retroviruses, and may therefore be used to generate libraries of insertion mutants. However, transposons are typically preferred to viruses because issues of safety related to pathogenicity may be avoided.

Transposons suitable for use in the invention comprise a recognition site for an RNA polymerase. Any suitable transposon may be used for the generation of transposon insertion libraries as long as it is modified by the inclusion of a recognition site for an RNA polymerase.

Suitable transposons for use in bacteria which can be modified to comprise a recognition site for an RNA polymerase include Tn3, yδ, Tn10, Tn5, TnphoA, Tn903, Tn9 17, Bacteriophage Mu and related viruses. Any of the above mentioned transposons may be used in a method of the invention.

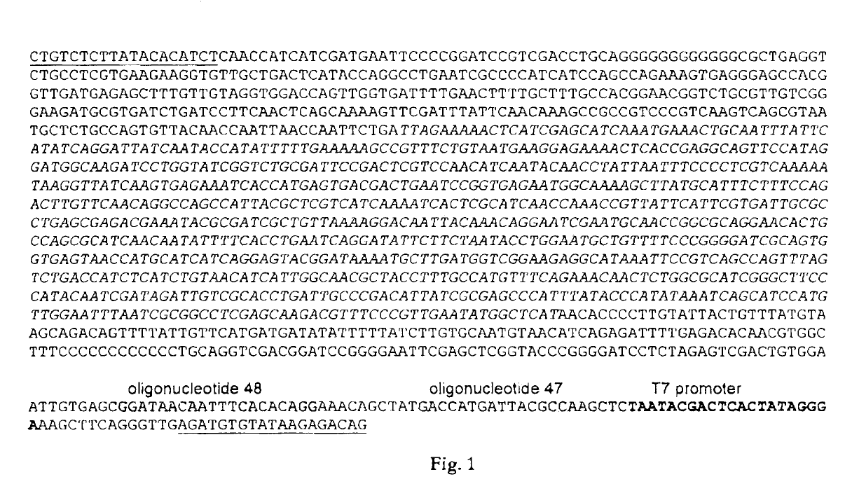

Preferred transposons are those which carry antibiotic resistance genes (which may be useful in identifying mutants which carry a transposon) including Tn5, Tn10 and TnphoA. For example, Tn10 carries a tetracycline resistance gene between its insertion site (IS) elements and Tn5 carries genes encoding polypeptides conferring resistance to kanamycin, streptomycin and bleomycin. A particular preferred transposon for use in the invention is a mini-Tn5 transposon, the sequence of which is set out in FIG. 1 and SEQ ID NO: 1.

It is of course possible to generate new transposons by inserting different combinations of antibiotic resistance genes between the IS elements or by altering the polynucleotide sequence of the transposon, for example by making a redundant base substitution in the coding region of an antibiotic resistance gene. It will be apparent that such transposons are included within the scope of the invention.

Suitable transposons for use in fungi which can be modified to comprise a recognition site for an RNA polymerase include the Ty1 element of Saccharomyces cerevisiae, the filamentous fungi elements (the filamentous fungi include agriculturally important plant pathogens such as Erysiphe graminis, Magnaporthe grisea) such as Fot1/Pogo-like and Tc1/Mariner-like elements (see Kempen and Kuck, 1998, Bioessays 20, 652-659 for a review of such elements).

Suitable transposons for use in plants which can be modified to comprise a recognition site for an RNA polymerase include Ac/Ds, Tam3 and other Tam elements, cin4 and spm.

Suitable transposons for use in animals which can be modified to comprise a recognition site for an RNA polymerase include P and hobo which may be used in Drosophila and Tc1 which can be used in Caenorhabditis elegans.

A transposon used in the invention comprises a recognition site for an RNA polymerase. Typically, the RNA polymerase recognition site points out of the transposon, i.e. so that transcription of sequence flanking the transposon insertion site may be transcribed. Preferred recognition sites are those for which the corresponding RNA polymerase is highly selective for initiation. Other preferred recognition sites are those for which the corresponding RNA polymerase does not Initiate transcription from sequences of the organism being studied. Preferred examples of RNA recognition sites are those recognised by bacteriophage RNA polymerases, for example the recognition site for T7 RNA polymerase, T3 RNA polymerase or SP6 RNA polymerase, in particular the T7 RNA polymerase recognition site. The recognition sites for these particular RNA polymerases are well known to those skilled in the art and the T7 RNA polymerase recognition site is shown in FIG. 1

The advantage of the use of an RNA polymerase recognition site in TMDH is that polymerases which are highly selective for transcription at their own promoter sequences and which do not initiate transcription for sequences in the host genome, for example the T7 RNA polymerase recognition site, can be used. Therefore, sequences in the RNA target sequences can only originate from sequences located downstream of the RNA polymerase recognition site located in the transposon and the possibility of transcription of essential gene sequences is reduced.

The RNA polymerase recognition site may appear anywhere within a transposon for use in the invention. However, typically the RNA polymerase recognition site will be located proximal to one end of the transposon, i.e. proximal to one IS. Typically, the 3′ end of the RNA polymerase recognition site will be situated from one to 30, for example from five to twenty base pairs away from the 5′ end of one of the IS. The RNA polymerase recognition site will typically point out of the transposon, i.e. towards the IS.

Libraries of transposon insertion mutants may be generated according to any method known to those skilled in the art. For example, libraries of bacterial transposon insertion mutants can be constructed using either plasmid or bacteriophage vectors containing the transposon and a selectable marker. Bacteriophage λ, eg. λTnphoA can be used to infect a suitable recipient bacterial strain, for example E. coli XAC. This E. coli strain has a suppressor mutation which prevents the bacteriophage from replicating and subsequently lysing and also contains an antibiotic resistance gene to allow selection of colonies containing transposed chromosomal DNA. The vector contains mutation(s) preventing integration of the λ chromosome into the bacterial host chromosome and thus the growth of false positive colonies without a mutated E. coli gene is prevented. Cultures of the recipient strain are grown in enriched medium (eg. Luria Broth) and cells in mid log phase of growth are infected with the λ transposon vector for 1 hour at 37° C. Aliquots of the infected cells are plated out on L-agar supplemented with the appropriate selective antibiotic and grown overnight at 37° C. These colonies constitute a transposon library and can be further analysed by the TMDH procedure described in this application.

Alternatively, transposasome complexes comprising the transposon in a complex with a transposase may be generated and electroporated into a suitable electrocompetent host. Suitable techniques for preparing transposasomes and for electroporating transposasomes into host cells are well known to those skilled in the art.

Growth of such libraries results in the generation of potentially thousands of insertion mutants all of which mutants carry insertion that are, of necessity, in genes that (when mutated) do not result in the death of the cell ie. are non-essential genes.

Each mutant in a suitable transposon insertion library may carry one transposon insertion. However, a mutant may carry more than one transposon insertion, for example two, three, four, five, ten or twenty transposon insertions. A transposon insertion mutant library suitable for use in the invention will comprise at least one transposon insertion mutant for at least 60%, at least 70%, typically at least 80%, preferably at least 90%, more preferably at least 95%, even more preferably at least 99%, or most preferably substantially all of the non-essential genes in the organism being studied. Preferably the library will be a saturating library, i.e. the library comprises a transposon insertion mutant for substantially all genes of the organism. A transposon insertion could be in an open reading frame of a gene or in a regulatory sequence of gene.

Any non-essential gene in the transposon insertion library may be represented by more than one insertion mutant, for example two, three, four, five or up to ten insertion mutants, each carrying transposon insertions at the same or different sites in the non-essential gene or carrying insertions at the same site in different orientations. Preferred libraries will have, on average, more than one different transposon insertion mutant for each non-essential gene represented in the library, for example at least two on average, at least four on average, at least 5 on average or at least 10 on average different transposon insertion mutants for each essential gene represented in the library.

Some regions of a particular genome may be inaccessible to insertion by a particular transposon, for example because of a particular secondary or tertiary structure which is inaccessible to a particular transposon. Thus it may be advantageous to combine two transposon libraries, thereby increasing the probability of obtaining transposon insertions in a greater number of genes. For example, in the case of bacterial libraries, Tn5 and Tn10 (both modified by the inclusion of an RNA polymerase recognition sequence) libraries for example, could be combined.

Generation of RNA Target Sequences

RNA target sequences are generated from the DNA (host organism) sequences that flank the transposons, i.e. non-essential gene sequences.

Generally, flanking sequence will be isolated from at least 60%, for example at least 70%, at least 80%, preferably at least 90%, more preferably at least 95%, even more preferably at least 99% or most preferably from substantially all of the transposon insertion mutants in a particular library of mutants.

In the method of the invention chromosornal DNA is prepared from the library of transposon insertion mutants. Techniques for the isolation of chromosomal DNA, alternatively referred to as genomic DNA, are well known to those skilled in the art.

The chromosomal DNA thus prepared is then digested with a restriction endonuclease. The restriction endonuclease is one which is capable of cutting at a recognition site which is located in the transposon at a position 5′ to the RNA polymerase recognition site and in the chromosomal DNA flanking the transposon 3′ to the RNA recognition site (which is in the transposon). The exact restriction enzyme to be used will depend on the sequence of the transposon. However, typically an restriction endonuclease is used which has recognition sites that appear frequently within the genome of the organism being studied. Thus, a series of DNA fragments is generated, some of which comprise an RNA polymerase recognition site fused to a portion of flanking sequence, i.e. non-essential gene sequence.

Generally, suitable restriction endonucleases will have six base pair, five base pair or preferably four base pair recognition sequences. Suitable examples of four base pair cutters are set out in Table 1 below:

TABLE 1

Examples of 4 bp recognition type II restxiction
endonucleases suitable for use in TMDH

| | | | |
|---|---|---|---|
| Enzyme | Recognition Site | Enzyme | Recognition Site |

| | | | | |
|---|---|---|---|---|
| Acil | C¹CGC | MseI | T¹TAA | |
| | GGC₁G | | AAT₁T | |
| AluI | AG¹CT | MspI | C¹CGG | |
| | TC₁GA | | GGC₁C | |
| BfaI | C¹TAG | NlaIII | ¹CATG | |
| | GAT₁C | | GTAC₁ | |
| BstuI | CG¹CG | RsaI | | GT¹AC |
| | GC₁GC | | CA₁TG | |
| DpnI | ¹GATC | Sau3a | ¹GATC | |
| | CTAG₁ | | CTAG₁ | |
| HaeIII | GG¹CC | TaqI | T¹CGA | |
| | CC₁GG | | AGC₁T | |
| HinpI | G¹CGC | Tsp509 | ¹AATT | |
| | CGC₁G | | TTAA₁ | |
| HhaI | GCG¹C | | | |
| | C₁GCG | | | |

The resulting fragments comprising the RNA polymerase site adjacent to non-essential gene sequence may then optionally be size selected. If size selection is carried out, fragments with a size of from about 100 bp to about 2000 bp or preferably of from about 200 bp to about 600 bp may be isolated, for example from a gel, and purified. The smaller the fragments isolated, the smaller the chance of the RNA target sequences including sequences from genes which lie next to genes which have been interrupted by transposons. If such adjacent sequences were from essential genes, there is the possibility that essential gene sequences could be identified as non-essential gene sequences. Thus, size fractionation can reduce the amount of false non-essential gene sequences.

The fragments comprising the RNA polymerase site fused to non-essential gene sequence are then self-ligated. Suitable techniques for carrying out self-ligation are well known to those skilled in the art. Any suitable ligase may be used, for example T4 DNA ligase. Ligation reactions may be carried out for from 6 to 24 hours, for example from 12 to 16 hours at a temperature of from 10° C. to 20° C., for example at about 16° C.

Self-ligated molecules are then amplified using iPCR. Techniques for carrying out iPCR are well known to those skilled in the art and may be carried out according to any suitable technique. Typically, iPCR is carried out using two oligonucleotides which bind divergently at a location 5′ to the RNA polymerase recognition site and 3′ to the restriction endonuclease recognition site in the transposon, i.e. the two olignucleotide recognition sites are located on the transposon between the restriction endonuclease recognition site and the RNA polymerase recognition site.

When using iPCR techniques, there is the possibility that, a “stuffer” fragment may ligate into the self-ligation reaction, which will be amplified along with the transposon-disrupted sequence. If this material were to be used in subsequent generation of the RNA target sequences, the stuffer sequence could create non-specific background signal as it would also be hybridized to the high density array. In order to remove this stuffer fragment, the sequences amplified in iPCR can be redigested with whichever enzyme was used to isolate the transposon-flanking sequence fragments in the first place. This results in the release of the stuffer fragments which can be removed from the transposon:flanking sequence fragment. Removal of the stuffer fragments can be facilitated if biotinylated primers are used in iPCR. The transposon-flanking sequence fragments can then be removed from the stuffer fragments using a magnetic-bead-streptavidin conjugate.

The transposon:flanking sequence fragments are then used to generate the RNA target sequences by carrying our in vitro transcription from the RNA polymerase recognition sequence. Techniques for carrying out in vitro transcription are will known to those skilled in the art and any suitable technique may be used. In essence, an RNA polymerase and ribonucleotides are used. Preferably, one or more of the ribonucleotides is labelled so that the resulting RNA target sequences are labelled. Suitable labels include radioactive labels, for example ³²P, ³³P or ³⁵S, or non-radioactive labels, for example an enzyme label, a fluorescent label or biotin.

Fluorescent labels are preferred, for example a water-soluble fluorescent dye such as Cy3™ or Cy5™ or a fluorescein-tagged compound such as FluorX™ (the NHS ester of carboxyfluorescein with an extended linker arm), fluorescein isothiocyanate (FITC) or 5-([4,6-Dichlorotriazin-2-yl]amino)fluorescein (DTAF).

Generally, it will only be necessary to have one of the four ribonucleotides labelled, although the use of more than one labelled ribonucleotide may allow greater signal intensity to observed when the RNA target sequences are hybridised with high.

The techniques described above allow the isolation of sequences flaking the transposons in a library of transposon insertion mutants. Thus, a pool of flanking sequences is generated collectively referred to as the RNA target sequences. Although fragments in the pool are generated from only one side of the transposons, a transposon is capable of inserting at any particular locus (that can be disrupted) in either orientation. Thus, in particular in a saturating transposon insertion library, many loci will be represented by mutants carrying insertions in both orientations. Therefore, the RNA target sequences generated according to the TMDH method of the invention will, for many loci, comprise flanking sequence in both orientations.

In a modification of the TMDH method, the chromosomal DNA isolated from a transposon insertion library may be divided into a number of aliquots. Those aliquots may then each be separately digested with a different restriction endonuclease which is capable of cutting at a recognition site which is located in the transposon at a position 5′ to the RNA polymerase recognition site and in the chromosomal DNA flanking the transposon 3′ to the RNA polymerase recognition site (which is in the transposon). The chromosomal DNA may be separated into, for example two, three, four, five or ten aliquots which are each separately digested with a different restriction endonuclease. Preferred restriction enzymes are as set out in Table 1 above.

Preferably, two aliquots of the chromosomal DNA are separately digested with different suitable restriction endonucleases, for example two of HaeIII, HhaI or Sau3AI. More preferably, three aliquots of the chromosomal DNA are separately digested with different suitable restriction endonucleases. If three aliquots are separately digested, preferably the three enzymes used are HaeIII, HhaI or Sau3AI.

If the TMDH protocol is used in this modified format, the different aliquots may be repooled after digestion and treated together in the subsequent steps of the method of the invention. Clearly, if this approach is used one pool of RNA target sequences is generated which is hybridised with an oligonucleotide array.

Alternatively, the digested aliquots may be treated separately in the subsequent steps. If this TMDH format is adopted, the resulting pools of RNA target sequences may be hybridised to the same or separate oligonucleotide arrays. Each pool of RNA target sequences may be labelled with a different, for example fluorescent, label. Thus, even if the separate pools of RNA target sequences are hybridised to the same oligonucleotide array, the hybridisation characteristics of each pool may distinguished from one another.

Hybridization of RNA Target Sequences to Polynucleotide Libraries

The pools of sequences which comprise the RNA target sequences may be used for hybridisation with olignucleotide arrays. Oligonucleotide arrays used in the TMDH protocol of the invention are preferably high density arrays.

Oligonucleotide arrays suitable for use in the invention may comprise sequences from one or more loci of a genome. Preferably suitable oligonucleotide arrays will represent (i.e. comprise at least one probe corresponding to) at least 80% of all open reading frames (ORFs), more preferably at least 90% of all ORFs, for example 95% of all ORFs, even more preferably 99% of all ORFs or substantially all ORFs of the genome represented on the oligonucleotide array.

By high density oligonucleotide array is meant an array in which there are a high number of probes covering the locus, loci or genome represented by the array. For example, in a high density oligonucleotide array there may be a probe, for example, for every 30 to 500 base pairs of the locus, loci or genome represented by the array. Preferably there will be a probe, for every 60 to 250 base pairs of the locus, loci or genome represented in the array, for example about every 100 base pairs. Probes may overlap, for example by 1, 2, 3, 4, 5, up to 10, up to 20, up to 30, up to 40 or up to 50 bases.

The oligonucleotide probes on the array are, for example, from about 9 to about 150 nucleotides in length, preferably from about 50 to about 100 nucleotides in length or more preferably about 60 nucleotides in length.

The oligonucleotide probes used in the array will typically be designed on the basis of the wild type sequence of the organism being studied. The oligonucleotide probes may be designed so that each probe has minimal or substantially no cross-hybridisation with other sequences in the genome from which the probes originate. The BLAST program can be used to design suitable probes (Altschul et al., J. Mol. Biol. 215, 403-410).

Methods for making oligonucleotide arrays are well known to those skilled in the art.

Probes which show no hybridisation or substantially no hybridisation (there may be a low level of background non-specific hybridisation) with the RNA target sequences are unlikely to have been disrupted by a transposon insertion event and consequently are strong candidates for sequences corresponding to essential genes.

However, it is theoretically possible for oligonucleotide probes within the 5′ or 3′-termini of essential genes to show a hybridisation signal with the TMDH protocol. For example, if a transposon insertion occurs in a non-essential gene adjacent to an essential gene, RNA target sequences may be generated from this transposon corresponding to both non-essential and essential gene sequences as a result of restriction sites lying within the essential gene. The resulting labelled target will not only comprise DNA corresponding to the non-essential gene (that has been disrupted), but will also extend into the adjacent essential gene up to the restriction site. The result of hybridising this labelled target to the oligonucleotide array will be appear as “bleed through” of signal to probes on either the 5′ or 3′ end of the essential gene, up to the restriction site used for the TMDH protocol.

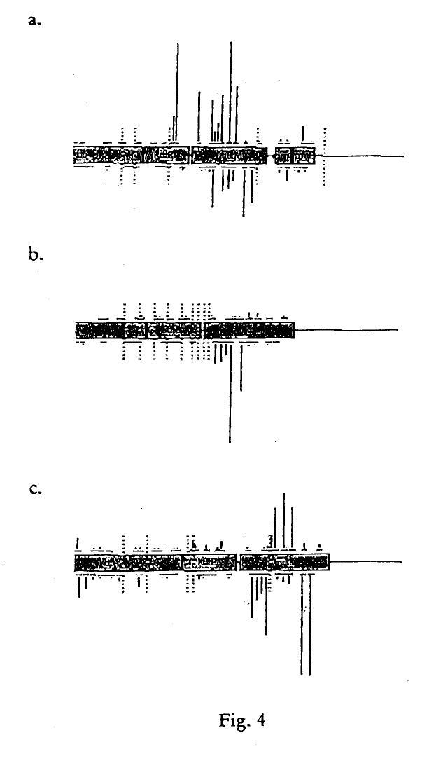

To address this potential source of mis-assignment of essential genes, TMDH may be carried out with more than one restriction endonuclease, for example two or three, which have with different recognition motifs. The more restriction sites that are used to generate target sequences, the more statistically unlikely it is that all of them will result in labelled RNA target sequences that “bleed through” into essential genes. The analysis of the resulting multiple array hybridisation patterns will remove any ambiguity on the site of transposon invention. An example of this resolution is shown in detail in the Example. The analysis of the fluorescence data from the three separate hybridisations is set out in FIG. 4. It is clear that the different restriction endonucleases used in the protocol for the generation of RNA target sequence give rise to different patterns of hybridisation. Furthermore, an analysis of the restriction map in context with the probe position allows the assignment of essentiality. For example, referring to FIG. 4, for all three restriction enzymes there is no hybridisation to internal probes within kdtB, supporting the idea that it is an essential gene. There is however a “bleed-through” signal using HaeIII on 3′-flanking probes up to a HaeIII site. Significantly, in the local genomic context, the next HaeIII site occurs 705 bp away in an adjacent non-essential gene, mutM, consequently a transposon insertion event between these sites will have occurred. Using this enzyme it will be possible to generate labelled target that will hybridise not only to the non-essential gene that has been disrupted, but also to extend to the adjacent essential gene. However, the data from the restriction enzymes Sau3AI and HhaI shows no ‘bleed-through’ from the adjacent non-essential gene. Overall, the combination of restriction sites, probe distribution on high-density arrays and hybridisation signal distribution can easily be analysed to determine the essential nature of the gene.

“Bleed-through” problems may also be addressed by isolating two separate pools of RNA target sequences, one pool comprising RNA target sequences derived from sequences flanking the left hand sides of the transposons and the other pool comprising RNA target sequences flanking the right hand sides of the transposons. Here, the transposon used to generate the library of transposon insertion mutants will comprise more than one, typically two, RNA polymerase recognition sites. If two RNA polymerase recognition sites are used, which may be the same or different RNA polymerase recognition sites, one is proximal to one end of the transposon and the other is proximal to the other end of the transposon.

Two separate aliquots of the library of transposon insertion mutants are treated separately in this version of TMDH. One aliquot is digested with a restriction endonuclease capable cutting 5′ of one of the RNA polymerase recognition sites in the transposon and 3′ of that RNA polymerase recognition site in the chromosomal DNA flanking the transposon. The second aliquot is digested with a restriction endonuclease capable cutting 5′ of the other RNA polymerase recognition site in the transposon and 3′ of that RNA polymerase recognition site in the chromosomal DNA flanking the transposon. Thus, separate left-arm and right-arm pools of transposon:flanking sequence fragments may be generated. Two separate pools of RNA target sequences are then generated from the transposon:flanking sequence fragments as described above.

The resulting left-arm and right-arm pools of RNA target sequences may then be hybridized to the same oligonucleotide array or separately to identical copies of the same oligonucleotide array. If the two pools of RNA target sequences are labelled with a different label, the hybridisation characteristics of each pool may be distinguished, even if the two pools are hybridised to the same oligonucleotide array.

A probe on the oligonucleotide array which shows no hybridisation with RNA target sequences in both the left- and right-arm arrays is likely to correspond to an essential gene. However, because of “bleed-through”, small regions of essential gene sequence may be isolated in a pool of RNA target sequences where a transposon inserts close to the end of a non-essential gene which lies adjacent to an essential gene. Thus, essential genes may be capable of generating a small hybridisation signal on an array. However, such an essential gene will show hybridisation to a probe only with one of the right- or left-arm arrays. Therefore, an essential gene sequence corresponds to a probe to which at least one of the left- and right-arm pools of RNA target sequences does not hybridise.

Thus, “bleed-through” effects may be revealed by using either of the two variants of TMDH described above. The two variants may be combined to further reduce the chance of essential gene sequences being mis-assigned as non-essential.

Identification of Conditional Essential Genes

The TMDH method may also be used for the identification of conditional essential genes. Conditional essential genes are those which are not absolutely essential for bacterial survival, but are essential for survival in particular environments eg. survival in a host (in the case of a pathogenic bacterium) or survival at elevated temperatures. Such environments are known as conditional restraints.

In order to isolate conditional essential genes, a library of transposon mutants is generated under control conditions (eg. growth at 37° C. in complete media). The library of mutants is then subjected to some conditional restraint. For example, the library of mutants can be inoculated in a suitable host, if it is a pathogen. Alternatively, the library of mutants can be grown at an elevated temperature. After the library of mutants has been subjected to the conditional restraint it can be recovered.

The library of mutants that have been exposed to the conditional restraint will lack mutants which carry transposons in those genes essential for growth under the conditional environment.

The control and conditional restraint libraries can then be subjected to TMDH as described above. The two resulting RNA target sequence libraries may then be hybridised separately to high density oligonucleotide arrays. Alternatively they can be hybridised to the same array, if the control and conditional restraint libraries are differentially labelled for example.

Comparison of the results given with the control and the conditional restraint libraries will allow the identification of genes which permit survival in the conditional restraint. Genes identified as essential for survival in the conditional restraint library, but not identified as essential for survival under control conditions should represent genes that are essential for survival under the conditional restraint. In particular, probes which show hybridisation with RNA target sequences from the input library, but which show no hybridisation or substantially no hybridisation (there may be a low level of background non-specific hybridisation) with RNA target sequences from the output library, are unlikely to have been disrupted by a transposon insertion event in the output library and consequently are strong candidates for sequences corresponding to conditional essential genes. The same “bleed through” considerations apply as set out above.

In the case of the analysis of conditional mutations in a pathogen, a library of Salmonella typhimurium transposon mutants, for example, can be used to infect a mouse. Following infection, bacteria target to livers and spleens and the course of infection can be conveniently followed by performing viable bacterial counts on those organs. The bacteria recovered from the livers and spleens can be grown on suitable plates. In the case of the conditional restraint at elevated temperature, a transposon-tagged library can be grown at 42° C.

Other conditional restraints include growth of antibiotic resistant bacteria in the present of antibiotics. This may reveal genes which are essential for antibiotic resistance. Such genes would be targets for drugs with the ability to lower bacterial resistance to particular antibiotics. Organisms could be grown in the presence of carcinogens, UV or other agents that cause oxidative stress and thus genes that confer resistance to growth under those conditions may be identified.

Verification of the Phenotype

Potential essential gene sequences and conditional essential gene sequences identified by the TMDH strategy may be verified using a method based on allelic exchange. This technique is particularly suitable for analysis of bacterial genes. PCR primers can be used to generate left- and right-arm sequences corresponding to the target gene sequence and ligated with a kanamycin-resistance encoding gene cassette. The resulting cassette can be introduced into a suicide vector, for example a plasmid-based vector, which is unable to replicate in a host bacterium.

In the case of a candidate essential gene, the resulting construct can be introduced into the bacterial strain from which the candidate gene originates. If the target gene is essential, it should be impossible to isolate allelic-exchange mutants that have a disrupted version of the target gene. In the case of a candidate conditional essential gene, the essential gene can be introduced into the bacterial strain from which the candidate gene originates. Allelic-exchange mutants can be isolated and subjected to growth under the conditional restraint. If the candidate gene is a conditional essential gene, it should not be possible for the allelic-exchange mutants to survive under the conditional restraint.

Similar experiments may be performed for other organisms.

Bioinformatics

The use of bioinformatics may allow the rapid isolation of further essential and conditional essential genes. A gene identified in TMDH may be used to search databases containing sequence information from other species in order to identify orthologous genes from those species. Genes so identified can be tested for being essential or conditionally essential using the genetic techniques described above. For example, an E. coli gene is identified as essential using a method as described above. This may allow the identification of a putative orthologue from Salmonella. That Salmonella gene may be tested by allelic exchange and the construction of conditional mutants in Salmonella as described above. Further orthologues may be identified in more distantly related organisms, for example from Plasmodium species.

Suitable bioinformatics programs are well known to those skilled in the art. For example, the Basic Local Alignment Search Tool (BLAST) program (Altschul et al., 1990, J. Mol. Biol. 215, 403-410. and Altschul et al., 1997, Nucl. Acids Res. 25, 3389-3402.) may be used. Suitable databases for searching are for example, EMBL, GENBANK, TIGR, EBI, SWISS-PROT and trEMBL.

Organisms Useful in the Invention

Organisms that may be used in the invention are those for which it is possible to carry out transposon mutagenesis and thus, those that can give rise to a library of transposon mutants. Clearly, if the genome is bigger, more mutants will have to be produced in order to give a better chance of achieving saturation mutagenesis. Suitable organisms include prokaryotic and eukaryotic organisms. Suitable prokaryotes include bacteria. Preferred bacteria are those which are animal or human or plant pathogens.

The bacteria used may be Gram-negative or Gram-positive. The bacteria may be for example, from the genera Escherichia, Salmonella, Vibrio, Haemophilus, Neisseria, Yersinia, Bordetella, Brucella, Shigella, Klebsiella, Enterobacter, Serracia, Proteus, Vibrio, Aeromonas, Pseudomonas, Acinetobacter, Moraxella, Flavobacterium, Actinobacillus, Staphylococcus, Streptococcus, Mycobacterium, Listeria, Clostridium, Pasteurella, Helicobacter, Campylobacter, Lawsonia, Mycoplasma, Bacillus, Agrobacterium, Rhizobium, Erwinia or Xanthomonas. Examples of some of the above mentioned genera are Escherichia coli—a cause of diarrhoea in humans; Salmonella typhimurium—the cause of salmonellosis in several animal species; Salmonella typhi—the cause of human typhoid fever; Salmonella enteritidis—a cause of food poisoning in humans; Salmonella choleraesuis—a cause of salmonellosis in pigs; Salmonella dublin—a cause of both a systemic and diarrhoeal disease in cattle, especially of new-born calves; Haemophilus influenzae—a cause of meningitis; Neisseria gonorrhoeae—a cause of gonorrhoea; Yersinia enterocolitica—the cause of a spectrum of diseases in humans ranging from gastroenteritis to fatal septicemic disease; Bordetella pertussis—the cause of whooping cough; Brucella abortus—a cause of abortion and infertility in cattle and a condition known as undulant fever in humans; Vibrio cholerae—a cause of cholera; Clostridium tetani—a cause of tetanus; Bacillus anthracis—a cause of anthrax. Suitable eukaryotes include fungi, plants and animals. Preferred eukaryotes include animal or human parasites and plant pests.

Suitable fungi include the animal pathogens including Candida albicans—a cause of thrush, Trichophyton spp.—a cause of ringworm in children, athlete's foot in adults. Other suitable fungi include the plant pathogens Phytophthora infestans, Plasmopara viticola, Peronospora spp., Saprolegnia spp., Erysiphe spp., Ceratocystis ulmi, Monilinia fructigena, Venturia inequalis, Claviceps purpurea, Diplocarpon rosae, Puccinia graminis, Ustilago avenae.

Suitable animal parasites include Plasmodium spp., Trypanasoma spp., Giarda spp., Trichomonas spp. and Schistosoma spp. Other animal parasites include the various platyhelminth, nematode and annelid parasites.

Suitable plant pests include insects, nematodes and molluscs such as slugs and snails.

Suitable plants include monocotyledons and dicotyledons.

Preferred organisms are those for which the entire genome is known and for which it may be possible to construct a high density oligonucleotide array covering the entire genome or all of the open reading frames.

Screens for Inhibitors of Essential and Conditional Essential Genes

Essential and conditional essential genes of bacteria and the polypeptides which they encode may represent targets for antibacterial substances. Similarly essential and conditional essential genes of fungi and eukaryotic parasites, pests and plants and the proteins which they encode may represent targets for fungicides, antiparasitics, pesticides and herbicides respectively. Fungicides may have both animal and plant applications.

Furthermore, if a particular gene is essential or conditionally essential for a number of different bacteria, fungi, parasites, pests or plants, that gene and the polypeptide it encodes may represent a target for substances with a broad-spectrum of activity.

An essential or conditional essential gene identified by a method as described above and the polypeptide which it encodes may be used in a method for identifying an inhibitor of transcription and/or translation of the gene and/or activity of the polypeptide encoded by the gene. Such a substance may be referred to as an inhibitor of an essential or conditional essential gene. Thus, an inhibitor of an essential or conditional essential gene is a substance which inhibits expression and/or translation of that essential gene and/or activity of the polypeptide encoded by that essential or conditional essential gene.

Any suitable assay may be carried out to determine whether a test substance is an inhibitor of an essential or conditional essential gene. For example, the promoter of an essential or conditional essential gene may be linked to a coding sequence for a reporter polypeptide. Such a construct may be contacted with a test substance under conditions in which, in the absence of the test substance expression of the reporter polypeptide would occur. This would allow the effect of the test substance on expression of the essential or conditional essential gene to be determined.

Substances which inhibit translation of an essential or conditional essential gene may be isolated, for example, by contacting the mRNA of the essential or conditional essential gene with a test substance under conditions that would permit translation of the mRNA in the absence of the test substance. This would allow the effect of the test substance on translation of the essential or conditional essential gene to be determined.

Substances which inhibit activity of a polypeptide encoded by the essential gene may be isolated, for example, by contacting the polypeptide with a substrate for the polypeptide and a test substance under conditions that would permit activity of the polypeptide in the absence of the test substance. This would allow the effect of the test substance on activity of the polypeptide encoded by the essential or conditional essential gene to be determined.

Suitable control experiments can be carried out. For example, a putative inhibitor should be tested for its activity against other promoters, mRNAs or polypeptides to discount the possibility that it is a general inhibitor of gene transcription, translation or polypeptide activity.

Test Substances

Suitable test products which can be tested in the above assays include combinatorial libraries, defined chemical entities, peptide and peptide mimetics, oligonucleotides and natural product libraries, such as display (e.g. phage display libraries) and antibody products. Antibody products include monoclonal and polyclonal antibodies, single chain antibodies, chimaeric antibodies and CDR-grafted antibodies.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or stimulation tested individually. Test substances may be used at a concentration of from 1nM to 1000 μM, preferably from 1 μM to 100 μM, more preferably from 1 μM to 10 μM. Suitable test substances for inhibitors of essential or conditional essential genes include combinatorial libraries, defined chemical entities, peptides and peptide mimetics, oligonucleotides and natural product libraries.

The test substances may be used in an initial screen of, for example, ten substances per reaction, and the substances of batches which show inhibition tested individually.

Inhibitors of Essential Genes

An inhibitor of an essential or conditional essential gene is one which inhibits expression and/or translation of that essential gene and/or activity of the polypeptide encoded by that essential or conditional gene. Preferred substances are those which inhibit essential gene expression and/or translation and/or activity by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml^{31 1}, 500 μgml⁻¹, 1 mgml⁻¹, 10 mgml⁻¹, 100 mgml⁻¹. The percentage inhibition represents the percentage decrease in expression and/or translation and/or activity in a comparison of assays in the presence and absence of the test substance. Any combination of the above mentioned degrees of percentage inhibition and concentration of inhibitor may be used to define an inhibitor of the invention, with greater inhibition at lower concentrations being preferred.

Test substances which show activity in assays such as those described above can be tested in in vivo systems, such as an animal model of infection for antibacterial activity or a plant model for herbicidal activity. Thus, candidate inhibitors could be tested for their ability to attenuate bacterial infections in mice in the case of an antibacterial or for their ability to inhibit growth of plants in the case of a herbicide.

Therapeutic Use

Inhibitors of bacterial, fungal or eukaryotic parasite essential or conditional essential genes may be used in a method of treatment of the human or animal body by therapy. In particular such substances may be used in a method of treatment of a bacterial, fungal or eukaryotic parasite infection. Such substances may also be used for the manufacture of a medicament for use in the treatment of a bacterial, fungal or eukaryotic parasite infections The condition of a patient suffering from such an infection can be improved by administration of an inhibitor. A therapeutically effective amount of an inhibitor may be given to a human patient in need thereof Inhibitors of bacterial, fungal or eukaryotic parasite essential or conditional essential genes may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternlly, transdermally or by infusion techniques. The inhibitors may also be administered as suppositories. A physician will be able to determine the required route of administration for each particular patient.

The formulation of an inhibitor for use in preventing or treating a bacterial or fungal infection will depend upon factors such as the nature of the exact inhibitor, whether a pharmaceutical or veterinary use is intended, etc. An inhibitor may be formulated for simultaneous, separate or sequential use.

An inhibitor is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier. e.g. sterle water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an inhibitor is administered to a patient. The dose of an inhibitor may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

Live Attenuated Vaccines

The principle behind vaccination is to induce an immune response in the host thus providing protection against subsequent challenge with a pathogen. This may be achieved by inoculation with a live attenuated strain of the pathogen, i.e. a strain having reduced virulence such that it does not cause the disease caused by the virulent pathogen. Bacteria which carry mutations in conditional essential genes required for survival in a host isolated according to the methods described above may be good candidates for use in live attenuated vaccines.

The mutations introduced into the bacterial vaccine generally knock-out the function of the gene completely. This may be achieved either by abolishing synthesis of any polypeptide at all from the gene or by making a mutation that results in synthesis of non-functional polypeptide. In order to abolish synthesis of polypeptide, either the entire gene or its 5′-end may be deleted. A deletion or insertion within the coding sequence of a gene may be used to create a gene that synthesises only non-functional polypeptide (e.g. polypeptide that contains only the N-terminal sequence of the wild-type protein).

The bacterium may have mutations in one or more, for example two, three or four conditional essential genes. The mutations are non-reverting mutations. These are mutations that show essentially no reversion back to the wild-type when the bacterium is used as a vaccine. Such mutations include insertions and deletions. Insertions and deletions are preferably large, typically at least 10 nucleotides in length, for example from 10 to 600 nucleotides. Preferably, the whole coding sequence is deleted.

The bacterium used in the vaccine preferably contains only defined mutations, i.e. mutations which are characterised. It is clearly undesirable to use a bacterium which has uncharacterised mutations in its genome as a vaccine because there would be a risk that the uncharacterised mutations may confer properties on the bacterium that cause undesirable side-effects.

The attenuating mutations may be introduced by methods well known to those skilled in the art. Appropriate methods include cloning the DNA sequence of the wild-type gene into a vector, e.g. a plasmid, and inserting a selectable marker into the cloned DNA sequence or deleting a part of the DNA sequence, resulting in its inactivation. A deletion may be introduced by, for example, cutting the DNA sequence using restriction enzymes that cut at two points in or just outside the coding sequence and ligating together the two ends in the remaining sequence with an antibiotic resistance determinant. A plasmid carrying the inactivated DNA sequence can be transformed into the bacterium by known techniques such as electroporation or conjugation for example. It is then possible by suitable selection to identify a mutant wherein the inactivated DNA sequence has recombined into the chromosome of the bacterium and the wild-type DNA sequence has been rendered non-functional by homologous recombination.

The attenuated bacterium of the invention may be genetically engineered to express an antigen that is not expressed by the native bacterium (a “heterologous antigen”), so that the attenuated bacterium acts as a carrier of the heterologous antigen. The antigen may be from another organism, so that the vaccine provides protection against the other organism. A multivalent vaccine may be produced which not only provides immunity against the virulent parent of the attenuated bacterium but also provides immunity against the other organism. Furthermore, the attenuated bacterium may be engineered to express more than one heterologous antigen, in which case the heterologous antigens may be from the same or different organisms. The heterologous antigen may be a complete protein or a part of a protein containing an epitope. The antigen may be from a virus, prokaryote or a eukaryote, for example another bacterium, a yeast, a fungus or a eukaryotic parasite. The antigen may be from an extracellular or intracellular protein. More especially, the antigenic sequence may be from E. coli, tetanus, hepatitis A, B or C virus, human rhinovirus such as type 2 or type 14, herpes simplex virus, poliovirus type 2 or 3, foot-and-mouth disease virus, influenza virus, coxsackie virus or Chlamydia trachomatis. Useful antigens include non-toxic components of E. coli heat labile toxin, E. coli K88 antigens, ETEC colonization factor antigens, P.69 protein from B. pertussis and tetanus toxin fragment C.

The DNA encoding the heterologous antigen is expressed from a promoter that is active in vivo. Two promoters that have been shown to work well in Salmonella are the nirB promoter and the htrA promoter. For expression of the ETEC colonization factor antigens, the wild-type promoters could be used. A DNA construct comprising the promoter operably linked to DNA encoding the heterologous antigen may be made and transformed into the attenuated bacterium using conventional techniques. Transformants containing the DNA construct may be selected, for example by screening for a selectable marker on the construct. Bacteria containing the construct may be grown in vitro before being formulated for administration to the host for vaccination purposes.

The vaccine may be formulated using known techniques for formulating attenuated bacterial vaccines. The vaccine is advantageously presented for oral administration, for example in a lyophilised encapsulated form. Such capsules may be provided with an enteric coating comprising, for example, Eudragate “S” (Trade Mark), Eudragate “L” (Trade Mark), cellulose acetate, cellulose phthalate or hydroxypropylmethyl cellulose. These capsules may be used as such, or alternatively, the lyophilised material may be reconstituted prior to administration, e.g. as a suspension. Reconstitution is advantageously effected in a buffer at a suitable pH to ensure the viability of the bacteria. In order to protect the attenuated bacteria and the vaccine from gastric acidity, a sodium bicarbonate preparation is advantageously administered before each administration of the vaccine. Alternatively, the vaccine may be prepared for parenteral administration, intranasal administration or intramuscular administration.

The vaccine may be used in the vaccination of a mammalian host, particularly a human host but also an animal host. An infection caused by a microorganism, especially a pathogen, may therefore be prevented by administering an effective dose of a vaccine prepared according to the invention. The dosage employed will ultimately be at the discretion of the physician, but will be dependent on various factors including the size and weight of the host and the type of vaccine formulated. However, a dosage comprising the oral administration of from 10⁷ to 10¹¹ bacteria per dose may be convenient for a 70 kg adult human host.

Agricultural Use

Inhibitors of bacterial, fungal and pest essential or conditional essential genes may be administered to plants in order to prevent or treat bacterial, fungal or pest infections; the term pest includes any animal which attacks a plant. Thus inhibitors of the invention may be useful as pesticides. Inhibitors of plant essential or conditional essential genes may be administered to plants in order to reduce or stop plant growth, that is to act as a herbicide.

The inhibitors of the present invention are normally applied in the form of compositions together with one or more agriculturally acceptable carriers or diluents and can be applied to the crop area or plant to be treated, simultaneously or in succession with further compounds.

The inhibitors of the invention can be selective herbicides, bacteriocides, fungicides or pesticides or mixtures of several of these preparations, if desired together with further carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying active ingredients of the present invention or an agrochemical composition which contains at least one of the active ingredients is leaf application. The number of applications and the rate of application depend on the intensity of infestation by the pathogen. However, the active ingredients can also penetrate the plant through the roots via the soil (systemic action) by impregnating the locus of the plant with a liquid composition, or by applying the compounds in solid form to the soil, e.g. in granular form (soil application). The active ingredients may also be applied to seeds (coating) by impregnating the seeds either with a liquid formulation containing active ingredients, or coating them with a solid formulation. In special cases, further types of application are also possible, for example, selective treatment of the plant stems or buds.

The active ingredients are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the active ingredients and, where appropriate, a solid or liquid adjuvant, are prepared in known manner, for example by homogeneously mixing and/or grindingactive ingredients with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil, or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are nonionic, cationic and/or anionic surfactants having good emulsifying, dispersing and wetting properties. The term “surfactants” will also be understood as comprising mixtures of surfactants. Suitable anionic surfactants can be both water-soluble soaps and water-soluble synthetic surface-active compounds.

Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts, alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980. The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant.

Whereas conamercial products are preferably formulated as concentrates, the end user will normally employ dilute formulations.

The following Example illustrates the invention:

### EXAMPLE

Materials and Methods

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

E. coli M1655 (Blattner et al., Science 277(5331), 1453-1462, 1997) was grown on L-Agar (Sigma) and in L-Broth (Sigma). Tn5kan-T7 mutants of MG1655 were grown with the addition of 30 μg/ml kanamycin (Sigma). Oligonucleotides were synthesized by SIGMA-GENOSYS. The pMOD™<MCS> vector (pMOD) and transposase were purchased from EPICENTRE (Madison, Wis.).

Transposon Design

The mini-Tn5 vector pMOD was used as a substrate to generate the Tn5kan-T7 transposon (see FIG. 1). The T7 RNA polymerase (T7) promoter was amplified from pT7-Blue (Novagen) by PCR using:

| | |
|---|---|
| primer 67, | |
| 5′-CCGGCTCGTGTCGACTGTGGAATTG-3′; and | (SEQ ID NO:) |
| primer 68, | |
| 5′-CTGCAGGCATGCAAGCTTTCCCTATAG-3′. | (SEQ ID NO:) |

The resulting PCR product was digested with SalI and HindIII and cloned into the respective sites in pMOD. Subsequently, the kanamycin resistance marker from pUC4K (Pharmacia) was cloned into the EcoRI site yielding pMOD-Tn5kanT7.

Library Generation

Tn5kan-T7 transposon mutants were generated in E. coli MG1655 according to Epicentre protocols. Transposon Tn5kan-T7, constructed in the pMOD vector, was PCR amplified using the forward and reverse PCR primers (EPICENTRE) to achieve 0.1 μg Tn5kan-T7/μl. Transposasome complex generation was performed according to Epicentre protocols.

Electrocompetent MG1655 were generated by culturing bacteria to an OD₆₀₀ of 0.5 at 18° C. followed by three washes in 10% glycerol. The transposasome complex (1 μl) was electroporated into 70 μl of electrocompetent MG1655 (0.1 cm cuvette; 20 kV/cm, 25 μF, 200Ω) followed by outgrowth in 1 ml of SOC (Gibco) medium at 37° C. for 1 h. Subsequently, bacteria were plated onto L-agar plates containing kanamycin at 30 μg/ml to achieve 120 colonies per 90 mm Petri dish and incubated overnight at 37° C.

Bacteria on each plate (384 plates in total) were then re-suspended in 5 ml of L-Broth (20% glycerol) for archiving of stock libraries at −80° C. The total number of mutants generated was approximately 46,000. Chromosomal DNA from the sub-libraries was prepared from 1 ml of overnight L-broth cultures (inoculated with 10 μl of stock library grown at 37° C.) using a Qiagen DNeasy 96 Tissue Kit according to Qiagen protocols.

Amplification of DNA Flanking the Site of Transposition

(1) 3.75 μp DNA (187.5 ng) from each of the 384 chromosomal preparations was combined to generate 12 pools (comprising approximately 4000 mutants per pool) consisting of 6 μg/120 μl of DNA.

(2) Three sets of these pools were generated for separate digestion with either: (i) HaeIII; (ii) HhaI; or (iii) Sau3AI. DNA was digested overnight in 135 μl. Each pool was then passed through a Qiagen Gel Extraction column according to the manufacturer's instructions and eluted in 50 μl water.

(3) Self-ligation of 10 μl (1.2 μg) of the digested DNA in a final volume of 150 μl was performed overnight at 16° C. using Gibco T4 DNA ligase according to the manufacturer's instructions.

(4) Inverse PCR (iPCR), using the self-ligated chromosomal DNA as template, was performed on each of the ligated samples from the three separate digests to amplify the T7 promoter and the E. coli MG1655 DNA flanking the site of Tn5kan-T7 transposon insertion using:

| | |
|---|---|
| primer 47, | |
| 5′-GACCATGATTACGCCAAGCTC-3′; and | (SEQ ID NO:) |
| primer 48, | |
| 5′-CCTGTGTGAAATTGTTATCCG-3′. | (SEQ ID NO:) |
| ps | |

Target Generation

Fluorescently labelled RNA target was generated in an IVT reaction, using a T7 Megashortscript kit (Ambion) according to manufacturers instructions with the digested iPCR products as the template, using 2 μl of rCTP-Cy5 per 20 μl reaction.

Array Design

DNA microarrays based on the E. coli MG1655 genome sequence (Accession number U00096) were designed using the programme HOTSPOTS (Arrow Therapeutics Limited). This program contains algorithms that design 60-mer probes, with minimal cross-hybridisation profiles, approximately 100 bp apart, across the locus of interest.

Control probes, to both the sense and anti-sense strands, were designed to 33 genes known to be susceptible (i.e. to be non-essential genes) to Tn5kan-T7 transposition (yaiT, ybaJ, sfmD, appY, ompT, ydbE, ybdO, ycaK, ycdU, chaA, tdk, ycjG, ycjZ, ydbA_{—}2, ydeJ, b1696, yedU, glf, b2146, ompC, b2275, b2290, b2333, xapB, pitB, yqiG, b3050, yhaI, yhiS, hdeB, yhjY, yiaL, and yjcF).

Probes were also designed to the locus from rfaD-pyrE (25 genes; 3791614-3813395 bp), inclusive. This locus is known to contain both essential and non-essential genes (Belunis et al., J Biol Chem. 17;270 (46), 27646-52, 1995; Freiburg et al., J. Mol Microbiol. Biotechnol. 3(3), 483-9, 2001, Kupke et al., J. Biol. Chem. 275(41), 31838-31386, 2000; Austin et al., J. Bacteriol. 172(9), 5312-5325, 1990, Izard & Geerlof, EMBO J. 18(8), 2021-2030, 1999). Probes were also designed that contained base pair mismatches as a hybridisation stringency experiment. Arrays were synthesised in situ using an Agilent Inkjet Oligonucleotilde Array synthesiser (Arrow Therapeutics Limited).

Hybridisation and Washing Conditions

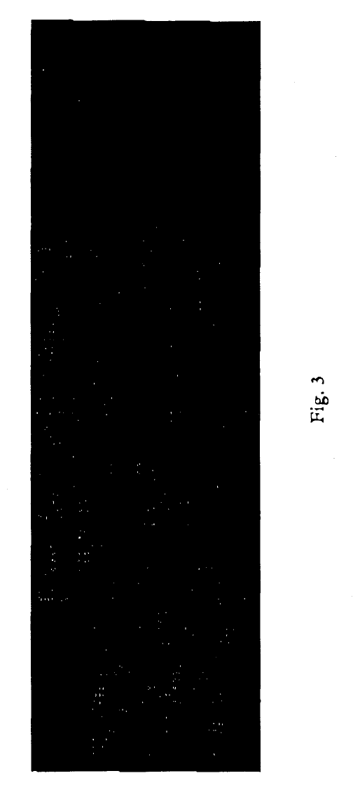

Fluorescently labelled target (25 μg) generated by IVT was diluted to 25 μl with water and heated to 70° C. for 5min, followed by snap chilling on ice. Target was then mixed with hybridisation buffer to a final volume of 280 μl (1× MES (100 mM 2-[N-Morpholino]ethane sulfonic acid; pH6.5-6.7), 20 mM EDTA, 1M NaCl, 1% Triton-X-100, 20% formamide) and hybridised ovemight at 55° C. Subsequently, the arrays were washed in 1× MES, 1M NaCl, 1% Triton-X-100 (45° C.) for 5 min and then in 1× MES, 0.1M NaCl, 1% Triton-X-100 (45° C.) for 5 min. Arrays were then rinsed in 1× MES, 0.1 M NaCl at room temperature. Arrays were then dried using a nitrogen gun and scanned on a micro-array scanner (Agilent) at 10 μm resolution (see FIG. 3).

Feature extraction of data from the arrays was preformed using Agilent software resulting in an Excel (Microsoft) formatted document containing probe position and signal intensity data. This data was then analysed using software generated at Arrow Therapeutics Limited (see FIG. 4).

Results and Discussion

Transposon Library Generation

A library of 46,000 Tn5kan-T7 mutants of E. coli MG]655 was generated. Assuming that Tn5kan-T7 transposes randomly in the MG1655 chromosome (comprising around 4000 genes) then non-essential genes in this library may be mutated around an average of 10 times. If the Tn5kan-T7 transposes at different sites in the gene, and in both orientations, then the target, generated following iPCR and IVT reactions from these random insertions, might be expected to hybridise to both sense and anti-sense probes, on an oligonucleotide array, designed over the whole length of a non-essential gene. Such data would allow unambiguous assignment of the gene as non-essential to E. coli viability. Correspondingly, genes that do not reveal hybridisation signatures are unlikely to have had a transposon insertion, and therefore may be regarded as candidate genes essential for bacterial viability.

Oligonucleotide Array Design

In order to maximise the information content of this series of TMDH experiments, we have designed a novel oligonucleotide genome array comprising optimised 60-mer oligonucleotide probes, with minimal cross-hybridisation profiles, approximately 100 bp apart.

Control experiments

As a control for the experimental protocol, 33 Tn5kan-T7 mutants of E. coli MG1655 were generated and their sites of transposition in the genome identified by DNA sequencing (data not shown). These genes are therefore known to be susceptible to Tn5kan-T7 transposition and can be regarded as non-essential. Probes on the micro-array corresponding to these genes were therefore used as positive controls for non-essential genes, with target expected to hybridise to these probes. The rfaD-pyrE locus is known to contain genes to both essential (kdtA, kdtB, dfp) and non-essential genes and provides a suitable test for the stringency of essential gene identification using this technique.

The Identification of Essential Genes

Chromosomal DNA isolated from a pooled library of 46,000 E. coli MG1655 Tn5kan-T7 mutants was used as template to generate Cy5-labelled target RNA, following iPCR and IVT reactions. This target RNA corresponds to the DNA flanking the sites of transposition. The iPCR reactions were carried out on DNA samples from the transposon library following digestion with three separate restriction digests (HaeIII, HhaI and Sau3AI). These enzymes were selected as they contain 4 bp recognition motifs and consequently are likely to maximise the recovery of templates for target generation from all of the mutants generated. The target generated following digestion with each of the enzymes was hybridised onto separate arrays, allowing a comparison to be made of the distribution of the signal.

Following hybridisation, the micro-arrays were scanned and the fluorescence output examined. The graphical output file for a hybridisation of the target resulting from a HaeIII protocol was over-layered with the gene positional information, highlighting the origins of the probes (see FIG. 3). FIG. 3 reveals that all the non-essential genes from yaiT-yjiK provided probes with saturating hybridisation intensity, with the majority having saturated probes over the whole length of the gene.

These data support the hypothesis that the library of 46,000 Tn5kan-T7 mutants contains multiple mutants in the 33 genes previously identified as non-essential and demonstrates that the TMDH technique is capable of the unambiguous identification of non-essential genes.

Analysis of the 25 genes in the rfaD-pyrE locus reveals that the majority of the probes have saturating levels of hybridisation, again with most genes having the majority of its probes binding to saturating amounts of target. This region of the genome contains several known essential genes, and if the TMDH protocol is working successfully, then these probes (corresponding to essential genes) should show no hybridisation signal. Analysis of the hybridisation pattern (FIG. 3) demonstrated that this was the case and probes to the known essential genes, kdtA and dfp, revealed no discernable hybridisation signal. The protein encoded by kdtA provides an essential role in LPS biosynthesis, while the protein encoded by dfp is thought to play a role in CoA biosynthesis.

Increased Resolution Using Three Restriction Endonucleases in TMDH

It is theoretically possible for oligonucleotide probes within the 5′ or 3′-termini of essential genes to show a hybridisation signal with the TMDH protocol. If a transposon insertion occurs adjacent to an essential gene and target is generated from this transposon as a result of restriction sites lying within the essential gene. The resulting labelled target will not only comprise DNA corresponding to the non-essential gene (that has been disrupted), but will also extend into the adjacent transposed essential gene up to the restriction site. The result of hybridising this labelled target to the oligonucleotide array will be appear as ‘bleed through’ of signal to probes on either the 5′ or 3′ end of the essential gene, up to the restriction site.

To address this potential source of mis-assignment of non-transposed candidate essential genes, TMDH is carried out with a panel of three restriction endonucleases with different recognition motifs. Consequently, it is statistically unlikely that all three enzymes will result in labelled target that ‘bleed through’ into essential genes, and the analysis of the resulting three array hybridisation patterns should remove any ambiguity. An example of this resolution is shown for the gene kdtB, which is known to be essential. The analysis of the fluorescence data from the three separate hybridisations can be tabulated, to show a graphical output of signal intensity. The annotated section of the locus shown in FIG. 4 also contains information on the sites of the restriction endonucleases used in the protocol and on the position of the oligonucleotide probes. It is clear that the different restriction endonucleases used in the target generation scheme give rise to different patterns of hybridisation. Furthermore, an analysis of the restriction map in context with the probe position allows the assignment of essentiality. For example, for all three restriction enzymes there is no hybridisation to internal probes within kdtB, supporting the idea that it cannot be transposed and is a candidate for classification as an essential gene. There is however a ‘bleed-through’ 3′ signal using HaeIII on 3′-flanking probes up to a HaeIII site. Significantly, in the local genomic context, the next HaeIII site occurs 705 bp away in an adjacent non-essential gene, mutM, consequently a transposon insertion event between these sites will have occurred. Using this enzyme it will be possible to generate labelled target that will hybridise not only to the non-essential gene that has been disrupted, but also to extend to the adjacent essential gene. However, the data from the restriction enzymes Sau3AI and HhaI shows no ‘bleed-through’ from the adjacent non-essential gene. Overall, the combination of restriction sites, probe distribution and hybridisation signal distribution can easily be assessed to determine the essential nature of the gene.

### SUMMARY

In summary, transposition of the Tn5kan-T7 transposon into the E. coli MG1655 chromosome enables a library of mutants to be generated which results in the T7 RNA polymerase promoter lying proximal to every site of transposition. The T7 promoter then permits the stringent generation of labelled target RNA in an IVT reaction. This RNA corresponds to the DNA sequences flanking each transposition insertion event. The resulting RNA target can then be labelled and hybridised onto an oligonucleotide array comprising an entire microbial genome allowing the identification of genes in the MG1655 genome that are susceptible (non-essential) to Tn5 transposition. Genes lacking a hybridisation signal can be unambiguously assigned and can be further examined as potential targets for anti-microbial drug discovery.

1. A method for identifying an essential gene of an organism, which method comprises:
(i) providing a library of transposon insertion mutants of the said organism, wherein the transposon comprises an RNA polymerase recognition site;
(ii) isolating chromosomal DNA from the library of (i);
(iii) digesting the chromosomal DNA with a restriction endonuclease that is capable of cutting 5′ of the RNA polymerase recognition site in the transposon and 3′ of the RNA polymerase recognition site in the chromosomal DNA flanking the transposon;
(iv) self-ligating the digested DNA;
(v) amplifying the self-ligated DNA by inverse PCR (iPCR);
(vi) transcribing RNA from the amplified DNA;
(vii) hybridising the transcribed RNA with an oligonucleotide array; and
(viii) identifying a probe on the oligonucleotide array which corresponds to an essential gene of the organism.

2. A method according to claim 1, wherein the amplified DNA produced in step (v) is digested with the same restriction endonuclease as used in step (iii) before being transcribed in step (vi).

3. A method according to claim 1, wherein the organism is a bacterium, yeast, fungus, plant or animal.

4. A method according to claim 1, wherein the transposon used in step (i) is a modified Tn5 transposon.

5. A method according to claim 4, wherein the RNA polymerase recognition site is situated proximal to one end of the transposon.

6. A method according to claim 1, wherein the RN9A polymerase recognition site is a T7 RNA polymerase or an SP6 RNA polymerase recognition site.

7. A method according to claim 1, wherein the restriction endonuclease in step (iii) has a four base pair recognition sequence.

8. A method according to claim 7, wherein the restriction endonuclease is HaeIII, HhaI or Sau3AI.

9. A method according to claim 1, wherein aliquots of the chromosomal DNA are digested separately with different restriction endonucleases in step (iii),
each of the restriction endonucleases being capable of cutting 5′ to the RNA polymerase recognition site in the transposons and 3′ to the RNA polymerase recognition site in the chromosomal DNA flanking the transposons and
each aliquot subsequently being treated separately in steps (iv) to (viii).

10. A method according to claim 9, wherein two aliquots of the chromosomal DNA are digested separately with different restriction endonuclease.

11. A method according to claim 10, wherein the two restriction endonucleases are two of HaeIII, HhaI or Sau3AI.

12. A method according to claim 9, wherein three aliquots of the chromosomal DNA are digested separately with different restriction endonucleases.

13. A method according to claim 12, wherein the three restriction endonuclease(s) are HaeIII, HhaI and Sau3AI.

14. A method according to claim 1, wherein two oligonucleotides which bind divergently to recognition sites 5′ to the RNA polymerase recognition site are used to carry out iPCR in step (v).

15. A method according to claim 1, wherein a labelled ribonucleotide is used in transcribing RNA from the amplified DNA in step (vi).

16. A method according to claim 1, wherein the separate aliquots are each transcribed using a different labelled ribonucleotide.

17. A method according to claim 16, wherein the separate aliquots are hybridised with the same oligonucleotide array in step (vii).

18. A method according to claim 1, wherein the oligonucleotide array comprises probes which are from 9 to 150 bp in length.

19. A method according to claim 1, wherein the oligonucleotide array comprises 1 probe for every 60 to 250 bp of the locus or loci represented on the array.

20. A method for identifying a conditional essential gene of an organism, which method comprises:
(a) providing a first sample of a library of transposon insertion mutants of the said organism (input library);
(b) providing a second sample of the library and subjecting that sample to a conditional restraint;
(c) collecting the mutants that survive the conditional restraint in step (ii) to give a second library (output library);
(d) carrying out a method according to steps (ii) to (vi) of claim 1 on the input library from step (a) and on the output library from step (c);
(e) hybridising the transcribed RNA derived from the input library and from the output library to the same or different oligonucleotide arrays; and
(f) identifying a probe on the oligonucleotide array(s) which corresponds to a conditional essential gene of the organism.

21. A method for identifying:
(i) an inhibitor of transcription and/or translation of an essential gene identified by a method according to claim 1 or a conditional essential gene identified by a method according to claim 20; and/or
(ii) an inhibitor of activity of a polypeptide encoded by a said gene,
which method comprises determining whether a test substance can inhibit transcription and/or translation of a said gene and/or activity of a polypeptide encoded by a said gene.

22. An inhibitor identified by a method according to claim 21.

23. An inhibitor according to claim 22, wherein the essential or conditional essential gene is from a bacterium, fungus or eukaryotic parasite.

24. A pharmaceutical composition comprising an inhibitor according to claim 23 and a pharmaceutically acceptable carrier or diluent.

25. A method of treating a host suffering from a bacterial, fungal or eukaryotic parasite infection, which method comprises the step of administering to the host a therapeutically effective amount of an inhibitor according to claim 23.

26. An inhibitor according to claim 22, wherein the essential or conditional essential gene is from a bacterium, fungus or pest.

27. A method of treating a bacterial, fungal or plant pest infection of a plant, which method comprises the step of administering to the plant an effective amount of an inhibitor according to claim 26.

28. An inhibitor according to claim 22, wherein the essential or conditional essential gene is a plant conditional or essential gene.

29. A method of inhibiting the growth of a plant, which method comprises the step of administering to the plant an effective amount of an inhibitor according to claim 28.

30. A method according to claim 20, wherein the organism is a bacterium and the conditional restraint is growth of that bacterium in its host.

31. A bacterium attenuated by a non-reverting mutation in one or more genes identified by a method as defined in claim 30.

32. A vaccine comprising a bacterium according to claim 31 and a pharmaceutically acceptable carrier or diluent.

33. A method of raising an immune response in a mammalian host, which method comprises the step of administering to the host a bacterium according to claim 31.

34. A method for the preparation of a pharmaceutical composition, which method comprises:
(a) identifying: (i) an inhibitor of transcription and/or translation of an essential gene or a conditional essential gene; and/or (ii) an inhibitor of activity of a polypeptide encoded by a said gene, by a method according to claim 21;
(b) synthesizing an inhibitor identified in step (a); and
(c) formulating the synthesized inhibitor with a pharmaceutically acceptable carrier or diluent.

35. A method of treating a host suffering from a bacterial, fungal or eukaryotic parasite infection, which method comprises:
(a) identifying: (i) an inhibitor of transcription and/or translation of an essential gene or a conditional essential gene; and/or (ii) an inhibitor of activity of a polypeptide encoded by a said gene, by a method according to claim 21;
(b) synthesizing an inhibitor identified in step (a);
(c) formulating the synthesized inhibitor with a pharmaceutically acceptable carrier or diluent; and
(d) administering to the host a therapeutically effective amount of the inhibitor formulated in (c).

36. A method for the preparation of a vaccine, which method comprises:
(a) identifying a conditional essential gene by a method according to claim 20;
(b) preparing a bacterium which comprises a non-reverting mutation in a conditional essential gene identified in step (a); and
(c) formulating the bacterium prepared in step (b) with a pharmaceutically acceptable carrier or diluent.

37. A method of raising an immune response in a mammalian host, which method comprises:
(a) identifying a conditional essential gene by a method according to claim 20;
(b) preparing a bacterium which comprises a non-reverting mutation in a conditional essential gene identified in step (a);
(c) formulating the bacterium prepared in step (b) with a pharmaceutically acceptable carrier or diluent; and
(d) administering to the host a bacterium formulated in step (c).


SEQUENCE LISTING (5 sequences)

SEQ ID NO: 1 - DNA, 1474 bp, Artificial Sequence
  Description of Artificial Sequence Tn5kan-T7 transposon
ctgtctcttatacacatctcaaccatcatcgatgaattccccggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatccggggaattcgagctcggtacccggggatcctctagagtcgactgtggaattgtgagcggataacaatttcacacaggaaacagctatgaccatgattacgccaagctctaatacgactcactatagggaaagcttcagggttgagatgtgtataagagacag

SEQ ID NO: 2 - DNA, 25 bp, Artificial Sequence
  Description of Artificial Sequence PCR primer
ccggctcgtgtcgactgtggaattg

SEQ ID NO: 3 - DNA, 27 bp, Artificial Sequence
  Description of Artificial Sequence PCR primer
ctgcaggcatgcaagctttccctatag

SEQ ID NO: 4 - DNA, 21 bp, Artificial Sequence
  Description of Artificial Sequence iPCR primer
gaccatgattacgccaagctc

SEQ ID NO: 5 - DNA, 21 bp, Artificial Sequence
  Description of Artificial Sequence iPCR primer
cctgtgtgaaattgttatccg